(19) United States
(12) Patent Application Publication
(10) Pub. No.: US 2011/0306930 A1
(43) Pub. Date: Dec. 15, 2011

(54) Hypodermic Needle

(75) Inventors: Gilbert Moersch, Stuttgart (DE); Franz Laermer, Weil Der Stadt (DE); Kathrin van Teeffelen, Stuttgart (DE); Christian Dorrer, Stuttgart (DE)

(73) Assignee: Robert Bosch GmbH, Stuttgart (DE)

(21) Appl. No.: 13/157,101

(22) Filed: Jun. 9, 2011

(30) Foreign Application Priority Data
Jun. 10, 2010 (DE) 102010029942.1

(51) Int. Cl. A61M 5/158; A61M 5/00

(52) U.S. Cl. 604/151; 604/272; 604/173

(57) ABSTRACT

A hypodermic needle, particularly for injecting a pharmaceutical substance into a human or animal body is disclosed. The needle provides a simple way of improving long-term use in a human or animal body. The hypodermic needle includes a tubular hollow body having a needle tip for introducing the hypodermic needle into the human or animal body, wherein the hollow body has a rigidity which is reducible by the influence of heat and/or moisture.

This application claims priority under 35 U.S.C. §119 to patent application no. DE 10 2010 029 942.1, filed Jun. 10, 2010 in Germany, the disclosure of which is incorporated herein by reference in its entirety.

### BACKGROUND

The present disclosure relates to a hypodermic needle, particularly for injecting a pharmaceutical substance into a human or animal body. The disclosure further relates to an injection assembly comprising the hypodermic needle.

For the subcutaneous and/or the intravenous administration of drugs from a drug delivery system, use is made of cannulas, such as hollow needles or hypodermic needles, through which in particular a liquid, such as a drug for example, can be introduced into the body. Classic hollow needles are made of metal and therefore only suitable to a limited extent for long-term use in body tissue. They are usually used for the intravenous delivery of drugs, but not for dosing into subcutaneous adipose tissue. In addition, these metal needles are suitable in particular for those sites at which the tissue is not excessively moved, stretched, or compressed, i.e. the arm for example. Furthermore, such needles are suitable only to a limited extent for long-term use, i.e. use in the body for a number of hours or days.

Plasters are already known which are usable in particular for treating acne. These plasters comprise small needles which dissolve in the body and, in doing so, release an active ingredient. Such plasters make it possible to release a limited amount of pharmaceutically active substance, more particularly solids.

Furthermore, drug delivery systems are known in which an elaborate injection principle is provided in or on the drug delivery system itself, or in which there is a specific piercing device with which a flexible tube can be introduced into tissue.

An object of the disclosure is to produce a hypodermic needle which provides a simple way of improving long-term use in a human or animal body.

### SUMMARY

The present disclosure provides a hypodermic needle, particularly for injecting a pharmaceutical substance into a human or animal body, comprising a tubular hollow body having a needle tip for introducing the hypodermic needle into the human or animal body, wherein the hollow body has a rigidity which is reducible by the influence of heat and/or moisture.

The hypodermic needle according to the disclosure is especially suitable for dosing drugs into subcutaneous adipose tissue, such as in the case of an insulin administration for example, at all sites of the body. It is hard and rigid enough in the original state to pierce skin, but then becomes soft and flexible in tissue and so does not disturb a person when moving.

Compared to conventional systems, the hypodermic needle according to the disclosure can be produced very easily and is, as a result, cost-effective. This is because, in the case of a drug delivery system, such as an insulin pump for example, it is possible to do without an elaborate injection device, such as a piercing aid for example. Furthermore, the disclosure provides the end user with very simple handling.

The initially hard or rigid hypodermic needle becomes less rigid, i.e. softer and/or more elastic, owing to the influence of moisture present in tissue and/or of body heat. As a result, the patient is not disturbed during any movement, such as during sport for example, or other daily situations, such as sleeping for example. It is advisable that at least the region of the hypodermic needle which is introduced into the body has a reducible rigidity.

Moisture means here the liquid present in tissue. This essentially means aqueous liquids, such as sodium chloride solutions for example, wherein of course liquids having a partial fat content also do not depart from the context of the disclosure.

For the purpose of the disclosure, rigidity of the hollow body or of the hypodermic needle means the resistance thereof to deformation or deflection. Suitable rigidities for introduction into the body are from 10 to 200 MPa, whereas a suitable rigidity of the hypodermic needle in the body is from 0 up to a few MPa, for example 5 MPa.

In the context of a preferred embodiment, the needle tip has a slanted region for perforating tissue upon introduction of the hypodermic needle into the human or animal body. As a result, introduction of the needle into the body makes a small cut in the tissue, whereby the insertion of the hypodermic needle causes distinctly less pain than, for example, that caused by a hypodermic needle which merely displaces the tissue.

In the context of a further embodiment, the hollow body is at least in part made of a material which comprises a matrix in which a water-soluble solid is arranged. The solid can thus impart to the matrix and therefore to the hypodermic needle in the default state, i.e. prior to introduction into the body, a rigidity that is sufficiently large for said needle to be introduced into the body without any problems. Owing to the influence of liquid or moisture present in the tissue, the solid is dissolved or dissolved out of the matrix, and so the rigidity of the hollow body is reduced and depends only on the matrix-comprising material.

In the context of a further embodiment, the hollow body is at least in part made of a material which comprises a matrix in which there is arranged a solid which melts or softens at or below the body temperature of the human or animal body. In this embodiment, the rigidity of the hollow body can be established by a solid which melts or softens owing to body heat, after the introduction of the hypodermic needle into tissue. This is based specifically on human body temperature, i.e. a value of about 37° C. Preferably, however, the melting point or, alternatively, the softening point is below 35° C. More particularly, the melting point or softening point is below 30° C.

As a result, rapid melting or softening can be achieved, which increases comfort for the patient. Furthermore, the hypodermic needle according to the disclosure deploys its activity as desired even if the body temperature, as usual, is subject to fluctuations or if the body to be treated is hypothermic.

Furthermore, it should be made sure that the melting point or softening point is not below room temperature. However, this may be necessary under certain conditions, in particular when the hypodermic needle is used for treating animals which often have a low body temperature. In this case, cooled storage of the hypodermic needles should be performed so that the desired rigidity is possible before introduction into the body.

In the context of a further embodiment, the hollow body is at least in part made of a swellable polymer. As a result, the moisture present in tissue can likewise be taken advantage of in order to reduce the rigidity of the hollow body. The polymer has sufficient rigidity in its default state, swelling up owing to the liquid or moisture in tissue and thus becoming softer and more supple.

In the context of a further embodiment, the hollow body has an inner layer and an outer layer, wherein the outer layer has a greater rigidity than the inner layer, and wherein the rigidity of the outer layer is reducible by the influence of heat and/or moisture. In this way, the rigidity of the hypodermic needle in its default state can be determined by the outer layer. After introduction into the body, the outer layer softens and so the rigidity is now determined by the inner layer. Thus, it is possible to use an inner layer which has exactly defined properties. These remain unchanged even during use, i.e. in the body, and are influenced or modified neither by factors prevailing in tissue, in particular heat and moisture, nor by the pharmaceutical substance to be delivered in the hypodermic needle.

It is particularly preferred for the outer layer to have a thickness of from 10 to 500 μm. Particularly advantageous here are thicknesses in the range from 100 to 200 μm. Such layer thicknesses can achieve sufficient rigidity, and equally a rapid reduction in the rigidity of the outer layer is possible in order to obtain as quickly as possible the desired properties in the body.

In the context of a further embodiment, the outer layer comprises a pharmaceutically active substance. Here, pharmaceutically active substances mean in particular anti-inflammatories, such as an antihistamine for example, or else analgesics. As a result, inflammations and the development of pain upon introduction of the hypodermic needle can be further avoided, which distinctly improves applicability.

The present disclosure further relates to an injection assembly, particularly for injecting a pharmaceutical substance into a human or animal body, comprising a base body having a topside and an underside, wherein there is arranged on the underside of the base body a multiplicity of hypodermic needles as described herein which have a fluid connection to a reservoir, arranged on the topside, for the pharmaceutical substance, wherein there is further provided at least one fastener to fasten the injection assembly to the human or animal body such that the hypodermic needles are introduced in the human or animal body.

Such an injection assembly is very simple to handle. The end user is able to fasten easily on the skin the assembly as a single unit and to inject in this way the hypodermic needles into the body at the same time. Elaborate tubing between the hypodermic needle, tube, and reservoir, as used for example in most conventional insulin pumps, is not necessary.

It is particularly advantageous for the reservoir to be connected to a pump and control unit which is arranged on the topside of the injection assembly. In this case, the system according to the disclosure has an especially compact size. Since the hypodermic needles are located directly on the delivery system, an extremely flat structure is possible. The patient is thus able to wear the system inconspicuously. In addition, the patient wears only the unit and does not have to carry the needle at one site of the body and the drug delivery system, connected by a tube, at another. As a result, wearing comfort is even further increased.

### BRIEF DESCRIPTION OF THE DRAWINGS

Further advantages and advantageous embodiments of the subjects according to the disclosure will be illustrated by the drawings and elucidated in the following description. It should be noted that the drawings are only descriptive in character and are not intended to restrict the disclosure in any way. In the figures,

FIG. 1 shows a schematic sectional view of one embodiment of a hypodermic needle according to the disclosure;

FIG. 2 shows a schematic sectional view of a further embodiment of a hypodermic needle according to the disclosure;

FIG. 3 shows a schematic side view of an injection assembly according to the disclosure;

FIG. 4 shows a schematic view diagonally from above of the injection assembly from FIG. 3.

### DETAILED DESCRIPTION

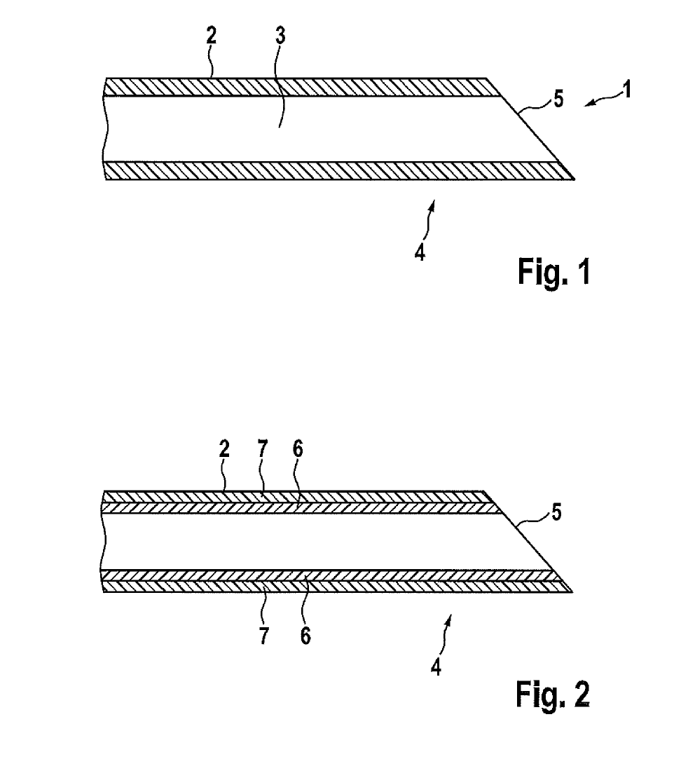

FIG. 1 shows one embodiment of a hypodermic needle 1 according to the disclosure. In the context of the disclosure, a hypodermic needle 1 means a cannula or a hollow needle which, for example, is used in medicine in particular for introducing liquids into a human or animal body or for removing liquids from the body. Therefore, the hypodermic needle 1 has in particular the purpose of injecting one or more pharmaceutically active substances or pharmaceuticals into a human body or into an animal body, i.e. of introducing them into the body. Hereinafter, the disclosure will be further described with respect to the body of a human patient, but will not be restricted thereto.

The hypodermic needle 1 according to the disclosure comprises a tubular hollow body 2. The tubular hollow body 2 has an inner space 3 through which the substance to be injected or to be removed is delivered into or out of the body. At one end region, the tubular hollow body 2 comprises a needle tip 4 which has the purpose of introducing the hypodermic needle 1 into the human or animal body. So that its introduction is achieved with minimal pain, the needle tip 4 preferably has a slanted region 5 for perforating skin and tissue when introducing the hypodermic needle 1 into the human body. Preferably, the slanted region 5 is also sharpened. In this way, during penetration of the tissue, a small cut can be made, which does not cause significant pain for the patient.

Opposite the needle tip 4, the hollow body 2 can, for example, be connected to a tube or to a syringe. For this purpose, the hollow body 2 can have a connector to which a syringe or else a tube can be attached. Furthermore, the hollow body 2 can be formed together with a tube as a single unit.

In order to penetrate human tissue, at least the needle tip 4 of the hollow body 2 has to be of sufficient rigidity. Preferably, however, the entire hollow body 2 is of sufficient rigidity to enable simple and problem-free introduction of the hypodermic needle 1 into the tissue of the body. However, the rigidity required for the introduction of the hypodermic needle 1 can, especially during prolonged use of the hypodermic needle 1, lead to discomfort and pain for the patient to be treated when, for example, he or she moves. In order to prevent this, the disclosure provides for the hollow body 2 having a rigidity which is reducible by the influence of conditions prevailing in the body or in the piercing region of the hypodermic needle 1, i.e. more particularly by the influence of heat and/or moisture.

Suitable materials for making the hollow body 2 are, for example, water-soluble and/or, more particularly, swellable polymers which are of sufficient rigidity in the default state, but become soft upon swelling through contact with moisture or liquid. Examples of such polymers are poly(2-hydroxyethyl methacrylate) (PHEMA), polyvinyl alcohol (PVA), or polydimethylacrylamide (PDMAA). Also possible is the use of polymers of crosslinked (co)polymerized hydrophilic monomers, graft (co)polymers of one or more hydrophilic monomers on a suitable graft base, crosslinked cellulose ethers or starch ethers, or natural products which are swellable in aqueous liquids, such as guar derivatives for example.

More particularly, the use of crosslinked polymers results in a hydrogel which becomes soft upon swelling. More particularly, preference is given to using PHEMA, since it is very hard in the dry state. However, other hydrogels as well can be used in the context of the disclosure.

Cellulose, for example, is suitable as a further possibility for making the hollow body 2. Cellulose is water-insoluble, but is extremely hygroscopic and so swells up when introduced to moisture since water molecules assemble between the cellulose fibers, and so becomes soft or increases in elasticity.

In a further embodiment, the hollow body 2 can at least in part be made of a material which comprises a matrix in which a water-soluble solid is arranged or finely distributed. More particularly, the material is constructed from a matrix which is porous and which is inherently soft and supple or elastic. Arranging a solid in the matrix allows the hollow body 2 to attain a rigidity which is sufficient for introduction of the hypodermic needle 1 in its default state into the body without any problems. The default state of the hypodermic needle 1 means here the state prior to the introduction of the needle into the body, i.e. at room temperature and normal humidity, or the state under defined storage conditions. When the hypodermic needle 1 is introduced into the body, it is acted on by an increased temperature, the body temperature, and also by increased moisture, viz. the moisture or liquid present in tissue.

More particularly, owing to the influence of the moisture, the solids distributed in the matrix can thus be dissolved and, in addition, possibly be removed from the matrix, leaving only the soft base material as the material providing rigidty. As a result, the hollow body 2 loses its rigidity or the rigidity is reduced. It now has the rigidity of the soft and supple matrix. It is advantageous here for the solid in the matrix to be very uniformly distributed. This enables a very uniform reduction in the rigidity of the hollow body 2 over its entire length or its entire dimensions.

The soft elastic matrix can, for example, be silicone. The distributed water-soluble solid should preferably not be harmful inside the body so that the body is not stressed as a result of the solid dissolving and, associated with this, the solid dissolving out of the matrix and thus of its distribution in the body. Examples of solids which can be used here are sugars, mineral salts, gelatin, or mixtures thereof.

In an alternative embodiment, the hollow body is again at least in part made of a material which comprises an especially porous and soft matrix. Instead of or in addition to the provision of a water-soluble solid in the matrix, it is equally possible, in order to impart sufficient rigidity to the hollow body 2 and thus to the hypodermic needle 1, to distribute or arrange a solid which melts or softens at or below the body temperature of the human or animal body. The outcome is that this solid melts or softens upon introduction of the hypodermic needle 1 into the body and of the body temperature acting as a result, whereby the rigidity of the hollow body and thus of the hypodermic needle 1 is reduced. Such solids can, for example, be fats or paraffins.

It is further conceivable for the hollow body to comprise mixtures of the abovementioned materials.

FIG. 2 shows a further embodiment of a hypodermic needle 1 according to the disclosure. The embodiment according to FIG. 2 likewise has a tubular hollow body 2 having a needle tip 4 which is preferably in the form of a slanted region 5 for perforation of tissue. However, according to FIG. 2, the tubular hollow body 2 has a multilayer construction. Particular preference is given to a construction composed of two layers. Therefore, the hollow body 2 of the injection needle 1 has an inner layer 6 and an outer layer 7, wherein the outer layer has a greater rigidity than the inner layer 6. The rigidity of the hollow body 2 in its default state is therefore essentially determined by the rigidity of the outer layer 7 and makes it possible for the hypodermic needle 1 to penetrate skin or tissue.

The inner layer 6 can then be a conventional, soft and elastic flexible tube, for example a Teflon tube, which is coated on its outer side by the outer layer 7.

In order to reduce the rigidity of the hollow body 2 and thus of the hypodermic needle 1 when the hollow body 2 is present in the body of a patient, the outer layer 7 can be formed such that its rigidity is reduced by the influence of heat and/or moisture. For this purpose, the outer layer 7 can, for example, comprise one material or a combination of materials, which material has been described with respect to FIG. 1.

It is further possible to form the outer layer 7 such that it is rigid in the original state, but, for example, dissolves completely owing to the influence of moisture, leaving only the flexible tube. The tube does not disturb the patient, even during long-term use. By way of example, the material here can be carboxymethyl cellulose (CMC), such as sodium CMC for example.

A further example of a coating forming such an outer layer 7 is a salt layer which is applied as an outer layer 7 to the inner layer 6, for example the Teflon tube. Such a salt layer can likewise provide suitable rigidity and is dissolved or removed owing to the influence of moisture inside the body. Salts which are suitable for this application are, more particularly, inorganic salts and organic salts. By way of example, calcium compounds, magnesium compounds, sodium compounds, or potassium compounds may be mentioned, such as sodium chloride, potassium chloride, sodium carbonate, potassium carbonate, sodium or potassium hydrogen carbonate, sodium or potassium gluconate, sodium or potassium citrate for example. The amounts of the salts used can be kept low in such a way that they also do not entail any adverse effects for the patient at the local level. Nevertheless, it may be advisable to watch out for potential secondary effects. For instance, in the case of a hypertensive patient for example, potassium chloride can be used instead of sodium chloride.

In general, the outer layer 7 can be used at a thickness of a few 10 μm, advantageously from ≧10 μm to ≦500 μm for example, more particularly from ≧100 μm to ≦200 μm. Firstly, this makes it possible to generate sufficient rigidity of the hollow body 2, enabling piercing of skin by the hypodermic needle 1 without any problems. Secondly, however, the concentration of the salts can be kept low in such a way that, in principle, an adverse effect on the body is minimized or ruled out.

A positive effect can be achieved when the outer layer 7 comprises one or more pharmaceutically active constituents. By way of example, inflammatory reactions or irritation of tissue can be prevented by anti-inflammatory agents. Examples of such active ingredients are antihistamines for example, more particularly nonsteroidal antihistamines, i.e. organic salts such as, for example, cetirizine HCl, diphenhydramine HCl or dimenhydrinate, an organic salt of diphenhydramine/8-chlorotheophylline. Furthermore, the treatment can be designed to be less painful by means of analgesic agents. Examples of usable analgesic agents are cocaine derivatives, such as lidocaine or novocaine for example. Furthermore, in addition to or instead of analgesic agents, the use of antibiotics against infections is also advantageous here.

The hypodermic needles 1 according to the disclosure can be used in a very wide variety of applications. More particularly, a hypodermic needle 1 according to the disclosure can be used in a drug delivery system and, here more particularly, in such a system in which long-term use is required, i.e. over a number of hours or a number of days for example. Such drug delivery systems are, for example, widespread in the case of insulin administration. The hypodermic needle according to the disclosure is then, for example, attached to a delivery system, such as an insulin pump for example.

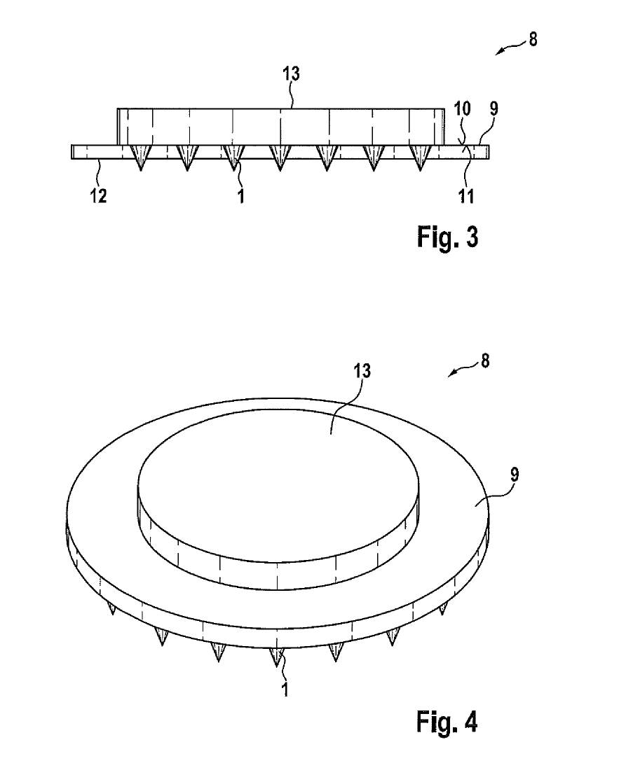

A further example of use is shown in FIGS. 3 and 4, which depict an injection assembly 8 according to the disclosure for injecting a pharmaceutical product into a human or animal body. The injection assembly 8 comprises a base body 9 having a topside 10 and an underside 11, wherein a multiplicity of hypodermic needles 1 according to the disclosure is arranged on the underside 11. On the underside 11, there is further provided one fastener or a multiplicity of fasteners to fasten the injection assembly 8 to the human or animal body. The fastener can preferably be an adhesive layer 12 arranged on the underside 11 of the base body 9. For this purpose, it is advantageous for the base body 9 to be made of a soft and supple material which can adjust to the surface contours of skin. As a result, the injection assembly 8 can be easily fastened to skin like a plaster, with the hypodermic needles 1 being introduced into the skin. In order to inject the appropriate active ingredient, there is arranged on the topside 10 a reservoir 13 for the pharmaceutical active ingredient, which reservoir has a fluid connection to the hypodermic needles 1.

So that delivery of the pharmaceutical active ingredient is exact and tailored to the needs of the patient, a pump and control unit is preferably arranged on the topside 10 of the base body 9 and connected to the reservoir 13. This pump and control unit can deliver the pharmaceutically active substance from the reservoir 13 into the body via the hypodermic needles 1 according to a predefined delivery schedule and can thus, for example, inject a defined amount per unit time. However, it is further possible to manually change the delivery schedule for example. For this purpose, the pump and control unit can preferably be connected wirelessly to an input device, or itself comprise an input device, with which a desired delivery schedule can be adjusted.

What is claimed is:

1. A hypodermic needle configured to inject a pharmaceutical substance into a human or animal body, comprising:
a tubular hollow body having a needle tip configured to introduce the hypodermic needle into the human or animal body,
wherein the hollow body has a rigidity which is reducible by the influence of heat and/or moisture.

2. The hypodermic needle according to claim 1, wherein the needle tip has a slanted region configured to perforate tissue upon introduction of the hypodermic needle into the human or animal body.

3. The hypodermic needle according to claim 1, wherein the hollow body is at least in part made of a material which comprises a matrix in which a water-soluble solid is arranged.

4. The hypodermic needle according to claim 1, wherein the hollow body is at least in part made of a material which comprises a matrix in which there is arranged a solid which melts or softens at or below the body temperature of the human or animal body.

5. The hypodermic needle according to claim 1, wherein the hollow body is at least in part made of a swellable polymer.

6. The hypodermic needle according to claim 1, wherein the hollow body has an inner layer and an outer layer, wherein the outer layer has a greater rigidity than the inner layer, and wherein the rigidity of the outer layer is reducible by the influence of heat and/or moisture.

7. The hypodermic needle according to claim 6, wherein the outer layer has a thickness of from 10 to 500 μm.

8. The hypodermic needle according to claim 6, wherein the outer layer comprises a pharmaceutically active substance.

9. An injection assembly configured to inject a pharmaceutical substance into a human or animal body, comprising:
a base body having a topside and an underside,
a multiplicity of hypodermic needles arranged on the underside of the base body, each of the multiplicity of hypodermic needles including a tubular hollow body having a needle tip configured to introduce the hypodermic needle into the human or animal body, wherein the hollow body has a rigidity which is reducible by the influence of heat and/or moisture,
a reservoir configured to contain the pharmaceutical substance, wherein the reservoir is arranged on the topside of the base body, and wherein each of the multiplicity of hypodermic needles has a fluid connection to the reservoir, and
at least one fastener configured to fasten the base body and the reservoir to the human or animal body such that the hypodermic needles are introduced in the human or animal body.

10. The injection assembly according to claim 9, further comprising a pump and control unit that is connected to the reservoir.

11. The hypodermic needle according to claim 7, wherein the outer layer has a thickness of from 100 to 200 μm.
